(19) United States
(12) Patent Application Publication
(10) Pub. No.: US 2019/0214134 A1
(43) Pub. Date: Jul. 11, 2019

(54) SYSTEM AND METHOD FOR AUTOMATED HEALTHCARE SERVICE

(71) Applicant: James Stewart Bates, Paradise Valley, AZ (US)

(72) Inventor: James Stewart Bates, Paradise Valley, AZ (US)

(21) Appl. No.: 15/863,078

(22) Filed: Jan. 5, 2018

(51) Int. Cl. G16H 50/20; G16H 10/60; G06N 5/02; G06N 99/00

(52) U.S. Cl. CPC G16H 50/20; G16H 10/60; G06N 3/0445; G06N 99/005; G06N 5/02

(57) ABSTRACT

Various embodiments of the present application relate to systems and methods for automated healthcare services, especially for automated healthcare services integrated with artificial intelligence. The automated healthcare system comprises a patient apparatus to provide symptom input, a medical information communication application to retrieve reference information with artificial intelligence integrated process, and a diagnostic engine to diagnose and present diagnostic results. The retrieved reference is mapped and categorized in a coherent way to provide support for the diagnostic engine. A guided measurement process may be incorporated for diagnostic assistance. The application of these methods may result in an improvement in efficiency and consistency for automated healthcare applications.

### BACKGROUND

### A. Technical Field

The present application relates generally to systems and methods for automatic healthcare services, especially for automated healthcare services capable of automatically retrieve reference and generating diagnostic information.

### B. Background of the Invention

With the healthcare industry's continuous driven for improving efficiency and throughput, automation of healthcare services becomes an important part of a strategy for performance improvement. Automation involves the application of control systems and information technologies to reduce the need for human work in the production of goods and services. Benefits provided by automated healthcare services include but not limited to labor savings, improved consistency, increased predictability, and high throughput, etc. Furthermore, automated healthcare services may generate lots of data which can be used for continuous performance optimization and improvement.

Among automated healthcare services, healthcare services integrated with artificial intelligence (AI) have become more and more popular. In healthcare application, AI uses various algorithms and software to approximate human cognition in the analysis of complex medical data to incrementally improve patient medical records, care delivery, diagnostic accuracy, and drug development, etc.

The implementation of AI not only improves care delivery, but also assists in clinician decision-making and operational efficiency, amplifying the impact of each individual practitioner. Although rapid progress has been made, various challenges still exist in implementing in automated healthcare, such as the requiring of robust dataset with both the breadth and depth for training in a particular application, patient health record recognition, and translation, incorporating latest medical developments, challenges in natural language processing, rare disease detection, etc.

What are needed are systems, devices and methods for automated healthcare services integrated with AI that can be smoothly implemented in retrieving reference and generating diagnostic information for clinician practices and patient care.

### BRIEF DESCRIPTION OF THE DRAWINGS

Reference will be made to exemplary embodiments of the present application that are illustrated in the accompanying figures. Those figures are intended to be illustrative, rather than limiting. Although the present application is generally described in the context of those embodiments, it is not intended by so doing to limit the scope of the present application to the particular features of the embodiments depicted and described.

FIG. 1 shows an exemplary automated healthcare system in accordance with various embodiments of the present application.

FIG. 2 shows an exemplary component diagram of a patient user apparatus in accordance with various embodiments of the present application.

FIG. 3 shows an exemplary modular diagram of an automated healthcare system in accordance with various embodiments of the present application.

FIG. 4 is an exemplary flow diagram illustrating a process of patient user authentication according to various embodiments of the present application.

FIG. 5 is an exemplary flow diagram illustrating a process for medical information communication application according to various embodiments of the present application.

FIG. 6 is an exemplary flow diagram illustrating a process of medical information translation for gathered medical information according to various embodiments of the present application.

FIG. 7 is an exemplary flow diagram illustrating a diagnosis process according to various embodiments of the present application.

FIG. 8 is an exemplary flow diagram illustrating a process of self- or assistant measurement or assistance measurement by the patient according to various embodiments of the present application.

FIG. 9 is a simplified block diagram of a computing device/information handling system according to various embodiments of the present disclosure.

One skilled in the art will recognize that various implementations and embodiments of the present application may be practiced in accordance with the specification. All of these implementations and embodiments are intended to be included within the scope of the present application.

### DETAILED DESCRIPTION OF THE PREFERRED EMBODIMENTS

In the following description, for purpose of explanation, specific details are set forth in order to provide an understanding of the present application. The present application may, however, be practiced without some or all of these details. The embodiments of the present application described below may be incorporated into a number of different electrical components, circuits, devices, and systems. Structures and devices shown in block diagram are illustrative of exemplary embodiments of the present application and are not to be used as a pretext by which to obscure broad teachings of the present application. Connections between components within the figures are not intended to be limited to direct connections. Rather, connections between components may be modified, re-formatted, or otherwise changed by intermediary components. Furthermore, one skilled in the art will recognize that embodiments of the present invention, described below, may be implemented in a variety of ways, such as a process, an apparatus, a system, a device, or a method on a tangible computer-readable medium.

Components, or modules, shown in diagrams are illustrative of exemplary embodiments of the invention and are meant to avoid obscuring the invention. It shall also be understood that throughout this discussion that components may be described as separate functional units, which may comprise sub-units, but those skilled in the art will recognize that various components, or portions thereof, may be divided into separate components or may be integrated together, including integrated within a single system or component. It should be noted that functions or operations discussed herein may be implemented as components. Components may be implemented in software, hardware, or a combination thereof.

Furthermore, connections between components or systems within the figures are not intended to be limited to direct connections. Rather, data between these components may be modified, re-formatted, or otherwise changed by intermediary components. Also, additional or fewer connections may be used. It shall also be noted that the terms “coupled,” “connected,” or “communicatively coupled” shall be understood to include direct connections, indirect connections through one or more intermediary devices, and wireless connections.

Reference in the specification to “one embodiment,” “preferred embodiment,” “an embodiment,” or “embodiments” means that a particular feature, structure, characteristic, or function described in connection with the embodiment is included in at least one embodiment of the invention and may be in more than one embodiment. Also, the appearances of the above-noted phrases in various places in the specification are not necessarily all referring to the same embodiment or embodiments.

The use of certain terms in various places in the specification is for illustration and should not be construed as limiting. A service, function, or resource is not limited to a single service, function, or resource; usage of these terms may refer to a grouping of related services, functions, or resources, which may be distributed or aggregated.

The terms “include,” “including,” “comprise,” and “comprising” shall be understood to be open terms and any lists the follow are examples and not meant to be limited to the listed items. Any headings used herein are for organizational purposes only and shall not be used to limit the scope of the description or the claims. Each reference mentioned in this patent document is incorporate by reference herein in its entirety.

Furthermore, one skilled in the art shall recognize that: (1) certain steps may optionally be performed; (2) steps may not be limited to the specific order set forth herein; (3) certain steps may be performed in different orders; and (4) certain steps may be done concurrently.

According to various embodiments, systems and methods for automated healthcare service are presented. In some embodiments, an automated healthcare system comprises a patient apparatus, a doctor apparatus, at least one self-measurement apparatus, a patient management server, computing apparatus, and a storage storing electronic medical record database. Those apparatuses are coupled wired or wirelessly via one or more networks for information exchange. Each of the apparatuses may be configured to comprise various functional components. Alternatively, some of the apparatuses may be integrated together as single equipment. Application software or APP may be installed on those apparatus to implement desired functionalities. One skilled in the art will recognize that these structural and functional components, described below, may be combined in various manners across embodiments of the present application.

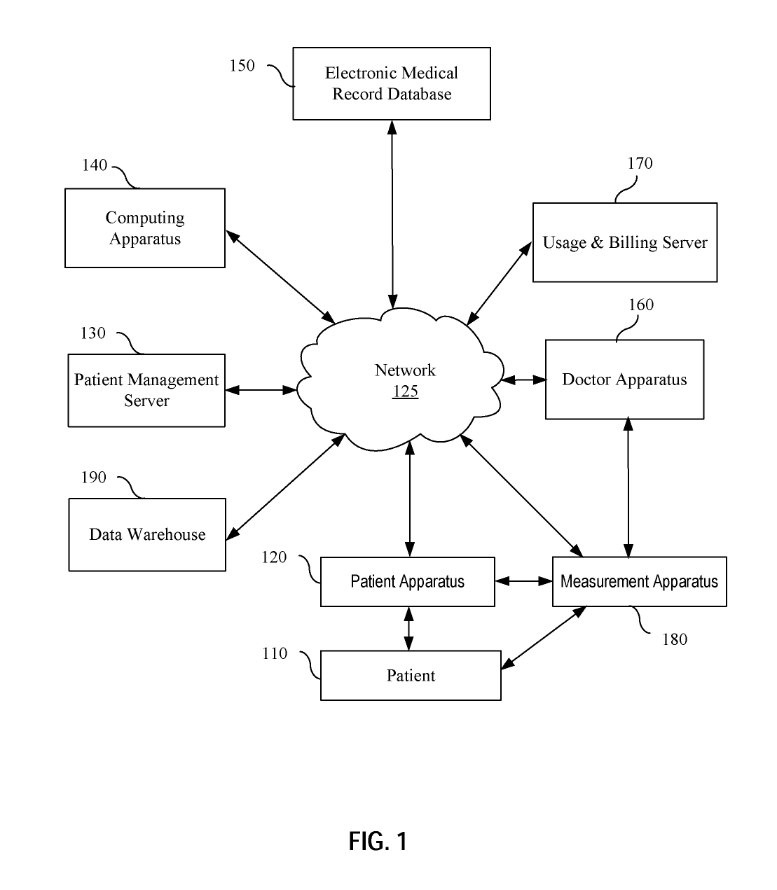

Embodiments of the present application will now be described in reference to FIGS. 1-9. Thereafter, specific embodiments of an automated healthcare service will be described with various structural and functional components. One skilled in the art will understand these embodiments may be implemented at various environments including, but not limited to, the hospital, senior care facility, nursing home, and recovery center, etc.

FIG. 1 shows an exemplary automated healthcare system in accordance with various embodiments of the present application. The automated healthcare system 100 comprises a patient apparatus 120, a patient management server 130, a computing apparatus 140, an electronic medical record database (such as internal EHR database or external EHR database) 150, a doctor apparatus 160, a usage/billing server 170, at least one measurement apparatus 180 (which may be a self-measurement apparatus or an assistant measurement apparatus operated by an assistant such as a nurse, a staff, an apparatus operator, or a layman with no prior medial knowledge or training, etc.) and a data warehouse 190 to store symptom, illness, treatment, outcome, medical coding data, and/or meta data for diagnosis and treatment analysis, etc. In embodiments, there are 4 sources of data in the warehouse 190. The first source is human entered relationship data, the second source is machine read textbook information, the third source is outside EMR patient machine learned data and the fourth source is AdviNow System used and machine learned data. The aforementioned apparatuses are coupled in wired or wireless links via one or more networks 125 for information exchange. Each of the apparatuses may be configured to comprise various functional components. Alternatively, some of the apparatuses may be integrated together as single equipment. Application software or APP may be installed on those apparatus to implement desired functionalities.

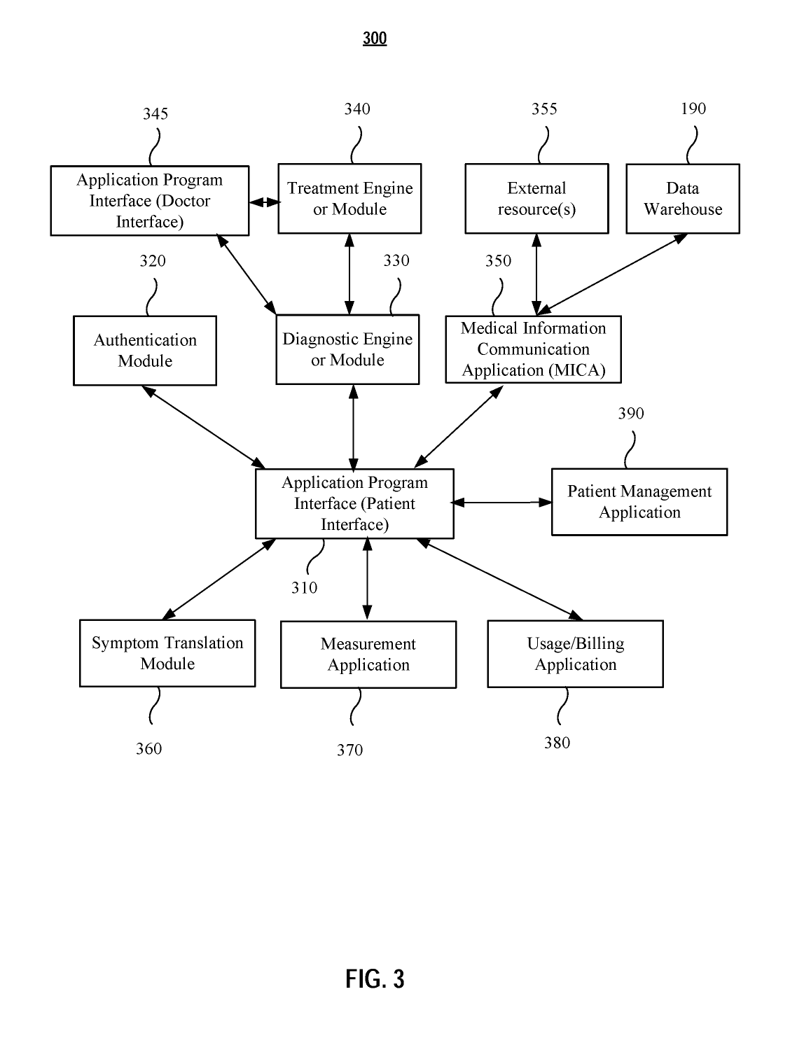

Both the patient apparatus 120 and the doctor apparatus 160 may be a tablet, computer, mobile device, or other electronic device, having an application program interface (API) for user interaction. The API may be a patient interface 310 (shown in FIG. 3), a doctor interface 345 (shown in FIG. 3), or a graphic user interface (GUI) configurable as various screen views to users (e.g., patients, doctors/nurses, operation managers, and administrators) based on user authorization level and other parameters. The API may interact with other components (e.g., touch screen, keyboard, and sensors) within the apparatus, and/or even other apparatus (e.g. the computing apparatus 140 and the patient management server 130, etc.). The API may provide audio/visual instructions on measurements (e.g., animation view and real time stream of the user).

In some embodiments, the patient apparatus 120 is a smart phone with a mobile app installed. The mobile app may be provided by the healthcare provider (such as a hospital) via a downloadable link. Once the patient registers or checked in, the downloadable link is sent to the patient's mobile phone. Afterwards, the patient is able to download, install and follow the instruction from the mobile app. In some embodiments, the patient apparatus 120 is a patient kiosk, which is installed at facility of the healthcare provider to provide self-guided service for the patient user. The Kiosk may integrate one or more management apparatus 180 for additional functionalities.

In operation, upon registration and authentication, a patient 110 may enter symptom-related data, such as type and duration of symptoms, health concerns, medical instrument measured diagnostic data, images, sound patterns, or other relevant information into the patient apparatus 120. The patient may use various ways of communication, such as voice control, to enter data, e.g., in the form of a machine-driven questionnaire. The patient apparatus 120 may communicate the data raw or in processed form with other apparatus via the network 125 in wired or wireless communication. The network 125 may be a local area network (LAN), a wide area network (WAN), a storage area network (SAN), or a combination thereof. The network 125 may be configured to support various communication methods, including but not limited to Wi-Fi, Ethernet, Bluetooth, optical fiber, etc.

In embodiments, the patient apparatus 120 may transmit the data in an encrypted format to other apparatuses via the network 125. The computing apparatus 140 may generate further request/question or diagnostic information based on the data received from the patient apparatus 120. The generation of further request/question or diagnostic information may be implemented using an artificial intelligence (AI) based process. The AI based process may involve software and/or hardware having a program of instructions embodied thereon, or a combination thereof. The generated request and/or question are transmitted back to the patient apparatus 120 for the patient to input more data accordingly. The generated diagnostic information is transmitted back to the doctor apparatus 160 for reference. In some embodiments, the computing apparatus 140 also search for a reference from the electronic medical record database 150 based on the data received from the patient apparatus 120. The reference may be health history of the patient, allergy history, background medical information related to the patient's input, common diseases related to patient's symptoms, common treatments related to patient's symptoms, latest medical discovery related to the patient's input, etc. In some embodiments, the reference is also generated using an AI based process. The AI process for the reference generation and the AI process for the generation of further request/question or diagnostic information may share same hardware, but use separate software modules. The computing apparatus 140 may be a centralized server or a cloud based server loaded with machine-executable instructions that may be in program modules that are executed by a processing device.

In embodiments, the patient management server 130 and usage/billing server 170 receive data from the patient apparatus 120 and/or the computing apparatus 140, such that the patient history can be updated and patient billing information may be generated accordingly. In embodiments, the computing apparatus 140 and the usage/billing server 170 also couples to the doctor apparatus 160, such that the doctor can access the information provided by these apparatuses. Furthermore, the time and cession usage from the doctor may also be tracked for record or billing purpose.

In embodiments, the computing apparatus 140, the patient management server 130 and usage/billing server 170 may be integrated as a single server to implement function of all three apparatuses. Furthermore, the electronic medical record database 150 may be stored within a memory of the single server. In other embodiments, the electronic medical record database 150 may be a third-party database accessible by the single server.

In embodiments, at least one measurement apparatus 180 couples to the patient apparatus 120 and/or the doctor apparatus 160. The patient is prompted via the patient apparatus 120 to use the measurement apparatus 180 for some self-measurement or assisted measurement in an effort to aid medical diagnosis. The measurement may be requested by a doctor, recommended by the computing apparatus 140 based on patient's input information, or by a routine check procedure. A software application may be implemented on the patient apparatus 120 to provide guidance to use the measurement apparatus 180. In some embodiments, various processes, including process with augmented reality, may be implemented to ensure that the measurement apparatus 180 is properly used. Gathered measurement data may be transmitted from the patient apparatus 120 and/or the measurement apparatus 180 to the computing apparatus 140 and the doctor apparatus 160 as reference for further reaction.

The measurement apparatus 180 is capable of measuring one or more characteristics of a patient. In embodiments, the measurement apparatus 180 is a combination of diagnostic medical devices that generate diagnostic data based on patient characteristics. Exemplary self-measurement apparatus may be heart rate sensors, otoscopes, digital stethoscopes, in-ear thermometers, blood oxygen sensors, high-definition cameras, spirometers, blood pressure meters, respiration sensors, skin resistance sensors, glucometers, ultrasound devices, electrocardiographic sensors, body fluid sample collectors, eye slit lamps, weight scales, and any devices known in the art that may be operated by the patient in performing a medical diagnosis. In embodiments, the patient apparatus 120 is preload with necessary application software such that the patient just plug-in and use the measurement apparatus 180. Alternatively, the measurement apparatus 180 may be a standalone apparatus for the patient to use. The gathered measurement data may be transmitted directly from the measurement apparatus 180, or via the patient apparatus 120, to other apparatuses, such as the computing apparatus 140 or the doctor apparatus 160.

In embodiments, the measurement apparatus 180 also comprises camera(s), proximity sensor, or other components to track the movement of the patient. Further, algorithms with augmented reality (AR) may be implemented to guide the patient to move the sensor to a desired target location for accurate and reliable measurements. Once the sensor, e.g., a stethoscope is placed at the desired target location on a patient's torso, the patient may be prompted by optical or visual cues shown on screen on the patient apparatus 120 or the measurement apparatus 180 to breathe according to instructions or perform other actions to facilitate medical measurements and to start a measurement.

In embodiments, the patient apparatus 120 and the doctor apparatus 160 are not located in the same facility. For example, a remotely located doctor may log in the automatic healthcare system 100 via the doctor apparatus 160 and provide instructions to the patient, e.g., by communicating via the network 125. Diagnostic results and treatment suggestions provided by the automated healthcare system 100 are accessible by the doctor for review, confirmation, or even modification if the doctor has different opinion. Input from the doctor may be transmitted back for further reaction, including treatment, billing, drug prescription, patient history update, etc.

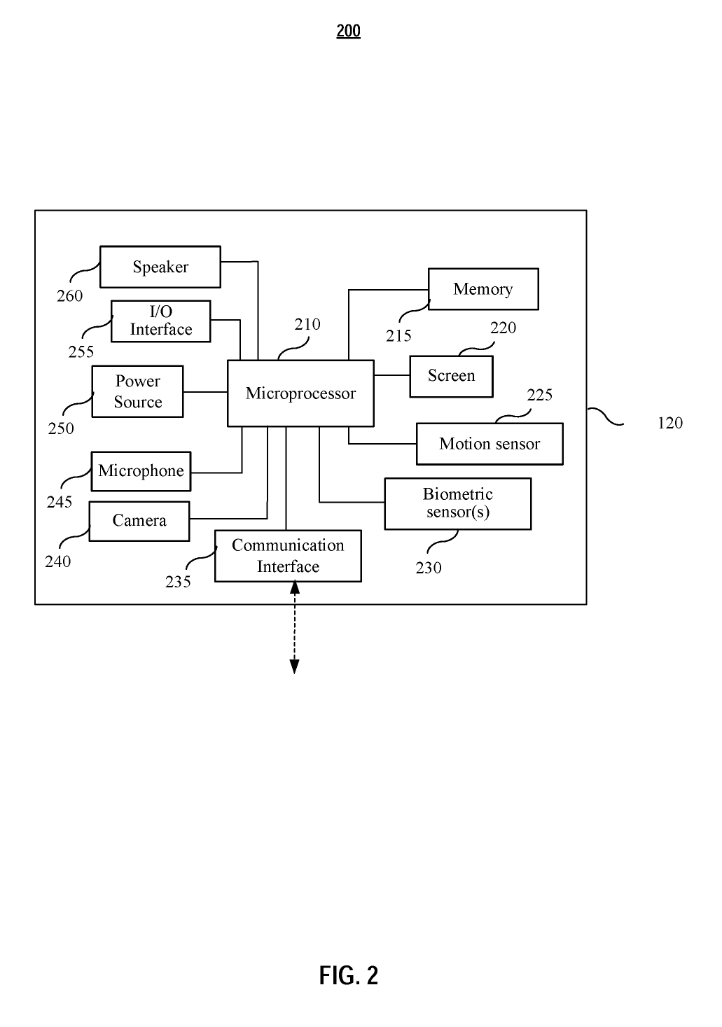

FIG. 2 shows an exemplary component diagram of a patient user apparatus in accordance with various embodiments of the present application. As depicted, the patient apparatus 120 comprises microprocessor 210, memory 215, screen 220, motion sensor 225, biometric sensor(s) 230, communication interface 235, camera 240, microphone 245, power source 250, I/O interface 255, and speaker 260. The patient apparatus 120 may further comprise additional components, may integrated with the one or more self-measurement apparatus. Some of the aforementioned components may be integrated into a single component. For example, the screen 220 and the I/O interface 255 may be integrated together into a touch screen.

In embodiments, the patient apparatus 120 securely communicates information in encrypted form to ensure privacy and the authenticity of patient input, as well as gathered measurement data from the measurement apparatus 180. Such encrypted communication may be accomplished by applying of security features embedded in hardware of the patient apparatus 120 and/or software that enable security features during transit and storage of sensitive data.

In embodiments, the communication interface 235 is an interface to enable bi-directional wired or wireless communications. For example, the communication interface 235 may transmit processed or raw data using various wireless communication protocol known in the art, such as Wi-Fi, Bluetooth, etc. For example, the communication interface 235 may receive instructions, in a text, audio or video format, for the patient to respond.

In embodiments, the patient apparatus 120 comprises at least one biometric sensor 230 to collect biometric information from the patient. The collected biometric information may be used for patient identification and/or authentication. The biometric sensor 230 may be a fingerprint sensor, an iris scanner, a DNA, etc. In some embodiment, the biometric sensor 230 is a camera or microphone. The patient apparatus 120 utilizes pre-loaded software to implement facial or voice recognition through the camera or microphone for identification and/or authentication.

In some embodiments, the patient apparatus 120 is a smart phone, a tablet, or a notebook, etc., with desired automatic healthcare service app installed. The app may be provided by the healthcare provider (such as a hospital) via a downloadable link, which is provided to the patient upon patient registration. After installation, an application program interface (API) is displayed on the screen of the patient apparatus 120 with instructions to guide the patient to start the automated healthcare service.

FIG. 3 shows an exemplary modular diagram 300 of an automated healthcare system in accordance with various embodiments of the present application. The application program interface (API) 310 displayed on the screen of the patient apparatus 120 is a specially designed interface portal that brings information from diverse sources together in a coherent and consistent way presentable to the patient. The diverse sources may be an authentication module 320, a diagnostic engine (or diagnostic module) 330, a treatment engine (or treatment module) 340, a medical information communication application (MICA) module 350, a symptom translation module 360, one or more measurement application 370 (which may be a self-measurement application or an assistant measurement application), and usage/billing application 380, and a data warehouse 190 to store symptom, illness, treatment, outcome, medical coding data, and/or meta data for diagnosis and treatment analysis, etc. In embodiments, there are 4 sources of data in the warehouse 190. The first source is human entered relationship data, the second source is machine read textbook information, the third source is outside EMR patient machine learned data and the fourth source is AdviNow System used and machine learned data. In embodiment, both the treatment engine 340 and the diagnostic engine 330 are coupled to a doctor interface 345 in a doctor apparatus 160 (not shown in FIG. 3). The modules, engines, or applications shown in FIG. 3 may be referred as software and/or hardware having a program of instructions embodied thereon, or a combination thereof.

In embodiments, the modules, engines, or applications shown in FIG. 3 are implementable at different apparatuses coupled to each other via the network 125 (not shown in FIG. 3). In embodiments, some of the modules, engines, or applications are implemented at the same apparatus. For example, the API 310 and the measurement application 370 may both be installed at the patient apparatus 120 (e.g. patient Kiosk). The diagnostic engine 330, the treatment engine 340, the MICA module 350, and the symptom translation module 360 may all be installed at the computing apparatus 140. The usage/billing application 380 may be installed together with the patient management application 390 in a hospital server.

In embodiments, the API 310 may further couples to an internal Electronic Health Record (EHR) database, which may be integrated within the patient management application 390. Input from the patient through the API 310, such as newly reported symptoms, updated drug history, etc., may be used to update the patient's internal EHR. The internal EHR is an electronic version of a patient's medical history, that is maintained and/or updated by the healthcare provider over time, and may include administrative clinical data relevant to the patient under the healthcare provider, including demographics, progress notes, problems, medications, vital signs, past medical history, immunizations, laboratory data and radiology reports. The internal EHR provides benefits to streamline the clinician's workflow. In embodiments, the internal EHR also has the ability to support other care-related activities directly or indirectly through various interfaces, including evidence-based decision support, quality management, and outcomes reporting. EHRs are important in the continued progress of healthcare that can strengthen the relationship between patients and healthcare providers. The data, and the timeliness and availability of it, would enable providers to make better decisions and provide better care.

A well-documented EHR can improve patient care by reducing the incidence of medical error by improving the accuracy and clarity of medical records, by making the health information available, reducing duplication of tests, reducing delays in treatment, and patients well informed to take better decisions, and/or reducing medical error by improving the accuracy and clarity of medical records.

In embodiments, the API 310 may further couple to MICA module 350 to fetch reference information from external resource(s) 355. The reference information may be external EHR of the patient, medical knowledge related to symptom input by the patient, reports on related symptoms, illness, or treatment, etc. The medical knowledge may include possible diagnosis and treatment solutions. The external resource(s) 355 may comprise external EHR database maintained by third-party healthcare providers, medical insurance database maintained by medical insurance company, medical knowledge database such as PubMed or Medline, etc. In embodiments, the possible diagnosis results and treatment solutions are ranked and only certain top diagnosis results and treatment solutions are transmitted back for the doctor's reference.

In embodiments, the reference information may also include information extracted from non-medical related sources, such as the social media, web post, etc. The reference information may include but not limit to life style information or analysis based on the patient's post on various social media. That reference information may be used by the diagnostic engine 330 for credibility check of the patient's input, and assistance in diagnostics, etc.

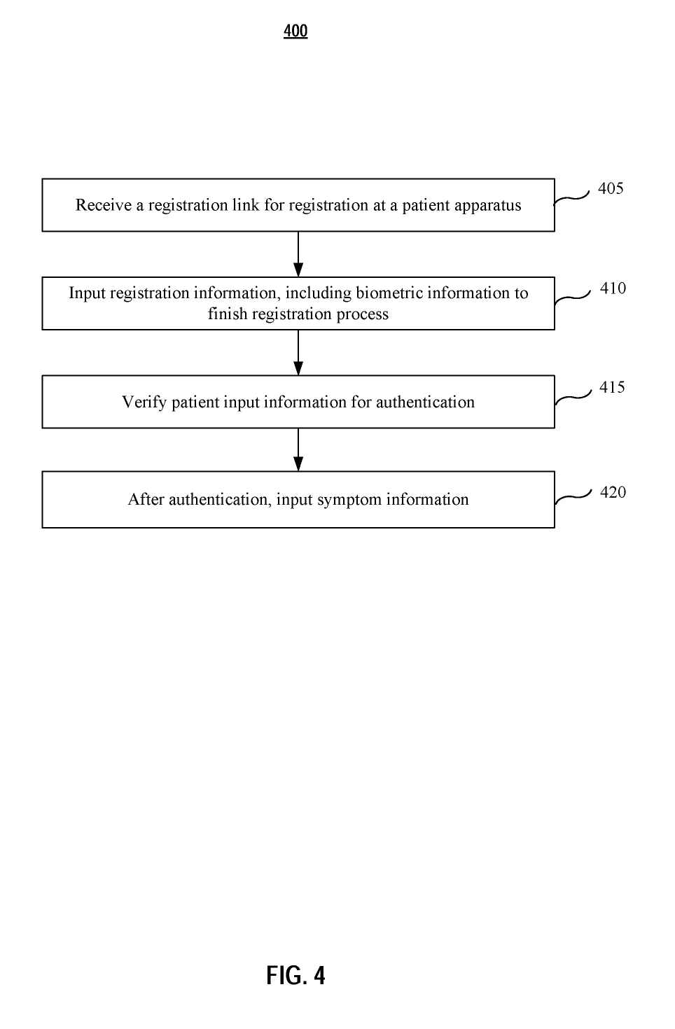

In embodiments, the diagnostic engine 330, the treatment engine 340, the MICA module 350, and the symptom translation module 360 may support AI algorithm integrated process and/or analysis in an effort to provide more accurate and convenient automated healthcare service to the patient. Some exemplary processes are disclosed in details with reference to FIGS. 4-8.

FIG. 4 is an exemplary flow diagram illustrating a process of patient registration and authentication according to various embodiments of the present application. One of ordinary skill in the art shall understand that the registration and authentication process may be done locally or remotely by a patient.

At step 405, a patient receives a registration link at a patient apparatus, which may be a smart phone. The registration link may be a download link for an application, such as a mobile app. Upon check-in, the registration link may be sent to the patient apparatus via email, text message, etc.

At step 410, after downloading and installing the app, an application program interface (API) is rendered on the screen of the patient apparatus. The API comprises instructions to guide the patient to input registration information to finish the registration process. The registration information may comprise name, social security, address, contract information, username, password, insurance information, health history such as allergy information, diseases history, etc. In some embodiments, the registration information may also include biometric information, such as facial photo, fingerprint or IRIS, etc., which may be collected during the registration process and will be used for patient recognition/authentication. The facial photo or fingerprint of the patient may be collected with the patient apparatus (such as a smart phone) conveniently. The IRIS may be scanned using an IRIS scanner, which may be a standalone apparatus or an attachment to the patient apparatus (for the scenario that the patient apparatus is a patient Kiosk).

In step 415, the registration information, including the biometric information, of the patient is transmitted via the network 125 to the patient management server to establish a patient record. Preferably, the information is securely transmitted (e.g. in an encrypted way) via the network 125.

For a returning patient, the process may start directly from step 420 with steps 405˜415 skipped. At step 420, the patient input information, such as username, password, fingerprint, etc., for authentication. The input information is compared to the established patient record. In embodiments, a patient may need to make a request to use the automatic healthcare service. Once the request is granted, a session link is sent to the patient apparatus. The patient clicks the link to start a service session beginning from the aforementioned authentication step 420.

In step 425, once the authentication passed, the patient may input symptom information to start the automatic healthcare service.

In some embodiments, when a hospital receives a patient who is in a coma condition and therefore not able to input information actively to begin the automatic healthcare service, a healthcare provider (such as a doctor, a nurse, or a first respondent, etc.) may help the patient to begin the process passively. For example, the healthcare provider may scan the patient's finger or IRIS to obtain biometric information, which may be transmitted to the hospital to search related internal EHR records and/or related reference information via the MICA interface. Any identified EHR record or reference information would be very beneficial in diagnostics and treatment.

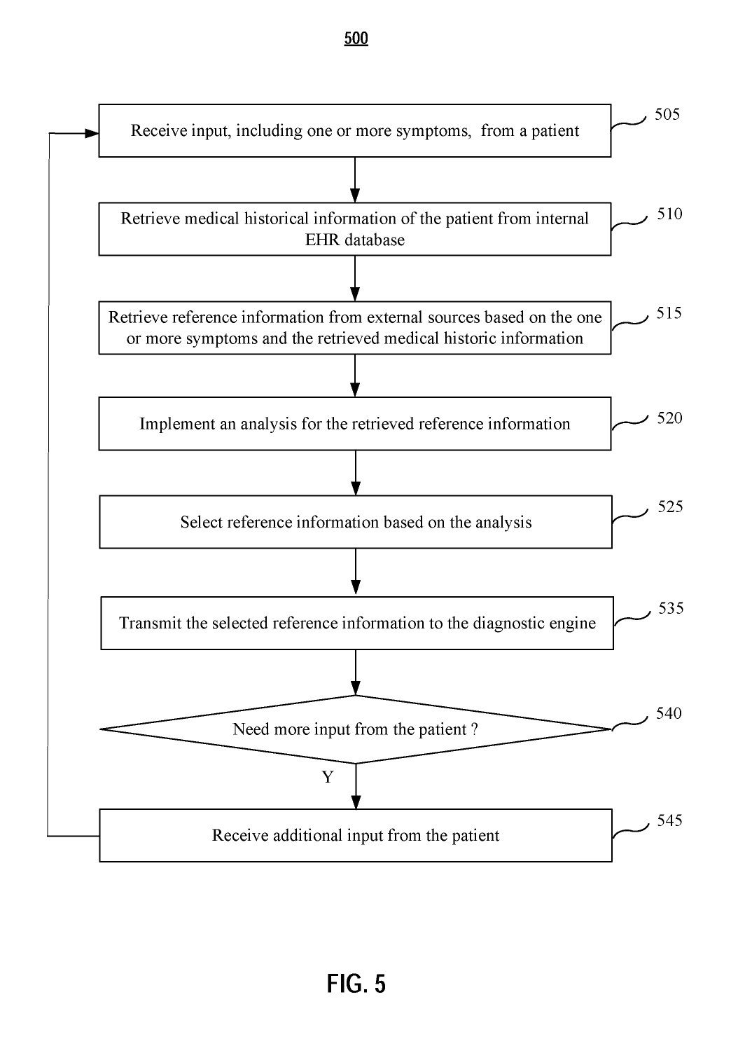

FIG. 5 is an exemplary flow diagram illustrating a process for medical information communication application (MICA) according to various embodiments of the present application. The MICA may be implemented at the computing apparatus, or a separate apparatus. Furthermore, the MICA module 350 may be configured support AI algorithm integrated process and/or analysis to provide more accurate reference information related to the patent's input symptoms.

As shown in FIG. 5, at step 505, patient's input, including one or more symptoms is received at the MICA. In embodiments, this step may be done after the registration or authentication process is finished. The input may be done by the patient via the patient apparatus. In embodiments, the initial input is transmitted to the MICA via a secure communication protocol for protection of privacy and integrity of the patient input data.

In embodiments, the one or more symptoms are received via voice input. The patient apparatus may be integrated with speech recognition module to translate the patient's audio input into text input. The speech recognition module may comprise artificial neural network such as recurrent neural network (RNN) or deep neural network (DNN), end-to-end automatic speech recognition module, connectionist temporal classification (CTC) based speech recognition module, etc. In embodiments, the speech recognition module may further comprise one or more language model to enable speech recognition in one or more languages.

At step 510, medical historical information related to the patient's input (or related to the patient) is retrieved from internal EHR database. In embodiments, the internal EHR database may be stored within the same apparatus with the MICA or stored in a separate server.

At step 515, the retrieved medical historical information and the one or more symptoms are used to retrieve reference information from external resource(s). In embodiments, the external resource may be external an EHR database maintained by other healthcare providers, or medical insurance company. The external resource may also be a medical knowledge database, such as Medline, a bibliographic database of life sciences and biomedical information including bibliographic information for articles from academic journals covering medicine, nursing, pharmacy, dentistry, veterinary medicine, and health care. The reference information may be retrieved via a third party search engine, such a Google, OmniMedicalSearch, MedNets, Hardin MD, Welch Medical Library, PDR.net, ClinicalTrials.gov, Intute, Healthline, PubMed, MedConnect, Entrez, eMedicine, MedBioWorld, Electronic Orange Book, PubGene, MDLinx, Medscape, etc. For example, PubMed is a search engine accessing primarily the MEDLINE database of references and abstracts on life sciences and biomedical topics. PubMed is maintained as part of the Entrez system of information retrieval by the United States National Library of Medicine (NLM) at the National Institutes of Health. In embodiments, the reference information may be retrieved via public health institute or organization, such as Centers for Disease Control and Prevention (CDC), World Health Organization (WHO), International Federation of Red Cross and Red Crescent Societies.

In embodiments, the reference information may be retrieved from non-medical external sources, such as the patient's social medial activities. These information may be very important in help diagnose possible diseases. For example, after a patient inputs symptom, the MICA found from the patient's social media posts that the patient recently travelled to a country and also found from a report issued by CDC that there was a pandemic spreading across the country. The MICA further found that the symptom reported by the patient is highly related to the pandemic according to a recently publish medical journal article. All above information may be extremely helpful in diagnostic possible disease of the patient. Without the information from the patient's social media, it might take much longer time or more efforts for the automatic healthcare system to identify the disease.

At step 520, the retrieved reference information is analyzed. In embodiments, an analysis is implemented for the retrieved information. The analysis may be a trustability analysis for all retrieved information, a contradictory analysis to identify contradictory information, and/or a ranking analysis by assigning different weight for retrieved information and ranking the information according to the assigned weight for each information, etc.

In embodiments, the trustability analysis comprises one or more from the following:

a) identifying source of each retrieved reference information;

b) identifying published date of each retrieved reference information;

c) identifying citation numbers of each retrieved reference information;

d) assigning a trustability factor to each retrieved information according to at least the source, published data, and the citation times;

e) ranking all retrieved reference information according to the assigned trustability factor;

f) implementing a conflict analysis to identify a conflict involving conflicted reference information among the retrieved reference information; and

g) solving the conflict by removing one or more prices if conflicted reference information from the identified conflicted reference information, the removed one or more conflicted reference information has lower trustability factor than remaining conflicted reference information.

For example, reference information A showing symptom A is related to disease A is found in more than 10 sources, including several top medical journals. While on the hand, reference information B showing symptom A is not related to disease A is only found ONCE from a medical journal with limited influence. Then reference information B will be removed to solve the conflict issue.

At step 525, based on the analysis at step 520, one or more reference information among all retrieved information is selected. In embodiments, the selection is based on the rank of the trustability factor and only top N reference information is selected, wherein N is an integer number, such as 5, 10, etc.

At step 530, the selected reference information is transmitted to the diagnostic engine for diagnosis.

In embodiments, the MICA process further comprises step 540 to determine whether more input from the patient is needed. The determination may be done by the diagnostic engine at the computing apparatus. The diagnostic engine evaluates the selected reference information and determines whether more input is needed. If yes, it generates a notice and transmits the notice back to the MICA. At the same time, the diagnostic engine may generate and transmits additional questions to be answers by the patient via the patient apparatus.

At step 545, the MICA receives further input from the patient. The process then goes back to step 505 for another round of reference retrieval and analysis.

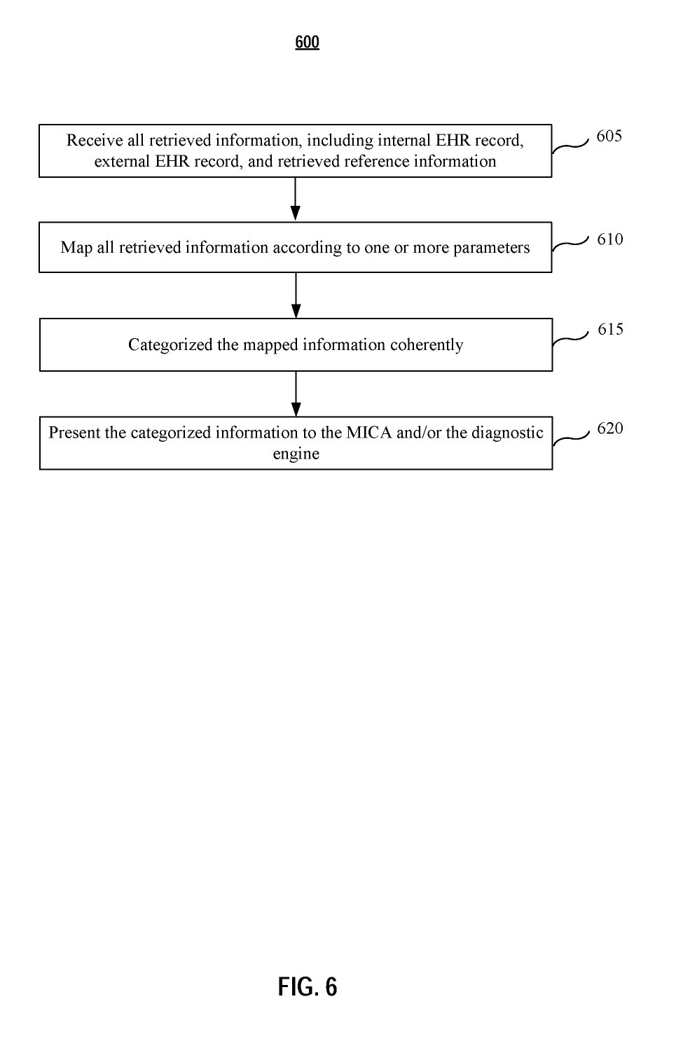

In embodiments, retrieved reference information, such as external EHR record, has different data format from internal EHR records. It would be confusing for the diagnostic engine to make proper evaluation if the format difference is not taken into consideration. In embodiments, the symptom translation module implement a symptom process to ensure that all retrieved information, including internal EHR information, external EHR information, as well as other reference information, are presented in a consistent and coherent way for diagnostics. FIG. 6 is an exemplary flow diagram illustrating a process of medical information translation for gathered medical information.

At step 605, the symptom translation module receives all retrieved information related to a patient and the patient's input. The retrieved information may include internal EHR record, external EHR record, retrieved reference information, etc. The retrieving process may be implemented by the MICA, as shown in FIG. 5.

At step 610, the retrieved information is mapped according to one or more symptom parameters, such as symptom type, duration of the symptom, and/or symptom intensity, etc. In embodiments, the mapping process may comprise a text recognition procedure and a group procedure. For example, some medical records (internal EHR or external EHR) may be a scan or photo without enough text annotation. A text recognition process may be implemented first for all those scan or photo documents to extracted text information, which may be used to describe those original documents. After the text recognition, all retrieved information is grouped according to one or more parameters. Each piece of information may belong to more than one group.

At step 615, all the mapped information is categorized such that the mapped information may be presented coherently in a consistent manner. For example, some medical records may adopt different scale systems to identify symptom parameters, such as pain intensity, discomfort degree, etc. If those different scale systems are not categorized coherently, the diagnostic engine may be misled by the information with various scale system. Various AI integrated algorithms may be implemented for coherent categorization. In embodiments, the categorization process may also comprise temporal categorization to identify chorological changes regarding the one or more parameters.

At step 620, the categorized information is presented or transmitted to the diagnostic engine for diagnosis or back to the MICA for further trustability analysis. In embodiments, the categorized information is securely transmitted in an encrypted format for data security and patient privacy.

One of ordinary skill in the art may understand that various processes may be implemented for symptom translation. Furthermore, in embodiments, the symptom translation process may be integrated within the MICA before step 520 such that the reference information are mapped and categorized coherently for more accurate trustability analysis.

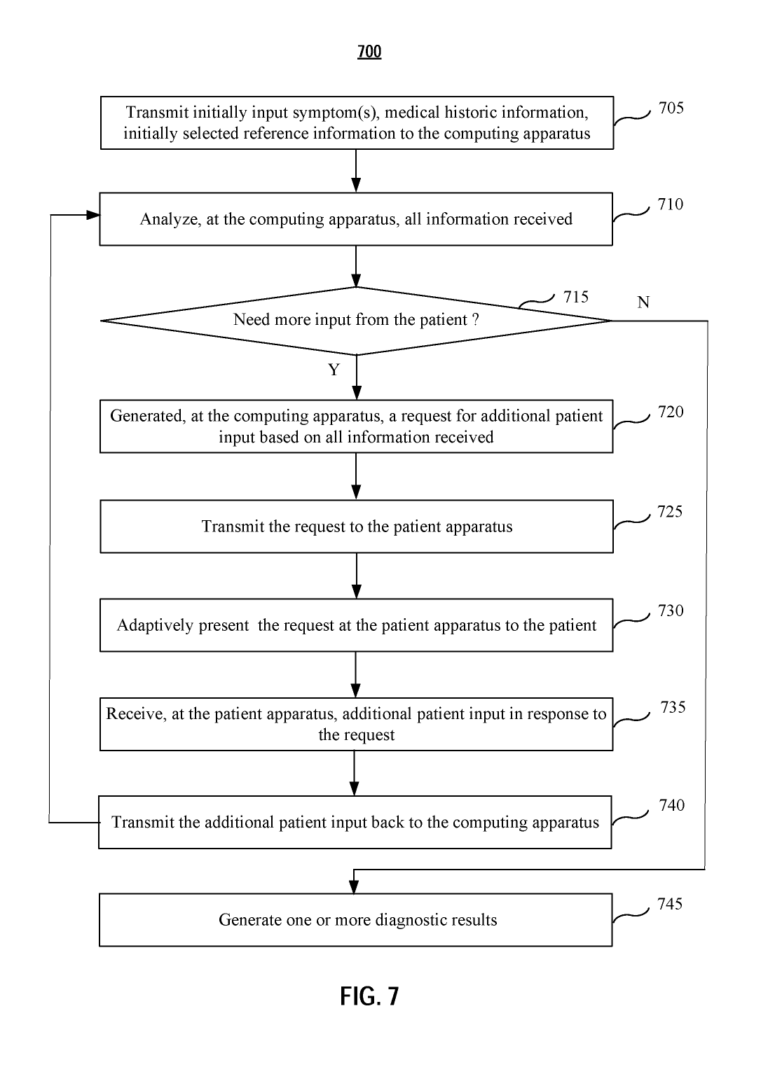

FIG. 7 shows an exemplary flow diagram illustrating a diagnostics process according to various embodiments of the present application. At step 705, the initial input symptom, medical historical information (including internal EHR and external EHR), and initially selected reference information are transmitted to the computing apparatus for the diagnostic engine to start the diagnostic process. The process of retrieving historical information and reference information may be referred to FIG. 5-FIG. 6.

At step 710, all the received information is analyzed at the computing apparatus. In embodiments, the diagnostic engine may comprise AI integrated algorithm and/or machine learning algorithms, such as recurrent neural network (RNN) or deep neural network (DNN), etc. In embodiments, the analysis process may comprise procedures of preprocessing, segmentation, region of interest analysis, evaluation, and classification, etc. In embodiments, the diagnostic engine may be pre-trained using known medical database before it is deployed online to improve the performance and efficiency.

At step 715, based on the analysis, a determination is made whether additional patient input from the patient is needed. If not, the process goes to step 745 directly, in which one or more diagnostic results are generated. In embodiments, the one or more diagnostic results are generated with each result associated with a possibility for doctor's reference.

If additional patient input from the patient is needed, the process goes to step 720, in which a request for additional patient input is generated at the computing apparatus. In embodiments, the request comprises one or more questions to be answered by the patient. In embodiments, the request asks the patient to use a self-measurement apparatus to obtain a self-measurement diagnostic result. In embodiments, the additional patient input is generated using AI integrated algorithm and/or machine learning algorithms. The one or more questions may be related to one symptom, such as whether the patient has a fever, how high the fever is, and how long the patient has been at the fever, etc.

At step 725, the generated one or more questions are transmitted to the patient apparatus. In embodiments, the questions are transmitted via a secure link in an encrypted format for data security and patient privacy.

At step 730, the generated one or more questions are adaptively presented to the patient based at least on retrieved patient medical history information. The questions may be adaptively presented in a text (with a specific size, font, color, etc.), audio, video, or a combination thereof. For example, if the patient medical history information shows that the patient had eye disease before, the generated one or more questions may be presented in an audio format, beside a text format. If the patient medical history information shows that the patient had hearing loss, the generated one or more questions may only be presented in a text format. If the patient medical history information shows that the patient is color blind, the generated one or more questions may be presented in a color that the patient is able to recognize.

At step 735, additional input with respect to the generated one or more questions are received at the patient apparatus. The additional input may be text input or voice input. The patient apparatus may be integrated with speech recognition module to translate the patient's audio input into text input. The speech recognition module may comprise artificial neural network such as recurrent neural network (RNN) or deep neural network (DNN), end-to-end automatic speech recognition module, connectionist temporal classification (CTC) based speech recognition module, etc. In embodiments, the speech recognition module may further comprise one or more language model to enable speech recognition in one or more languages.

At step 740, the additional input is transmitted back to the computing apparatus. The process then goes back to step 710 for a new ground of analysis for diagnosis.

In embodiments, the additional input may also be transmitted back to the MICA to retrieve additional reference information related to the additional input from the patient. The retrieved additional reference information is transmitted together with the additional input to the computing apparatus to begin the new round of analysis for diagnosis.

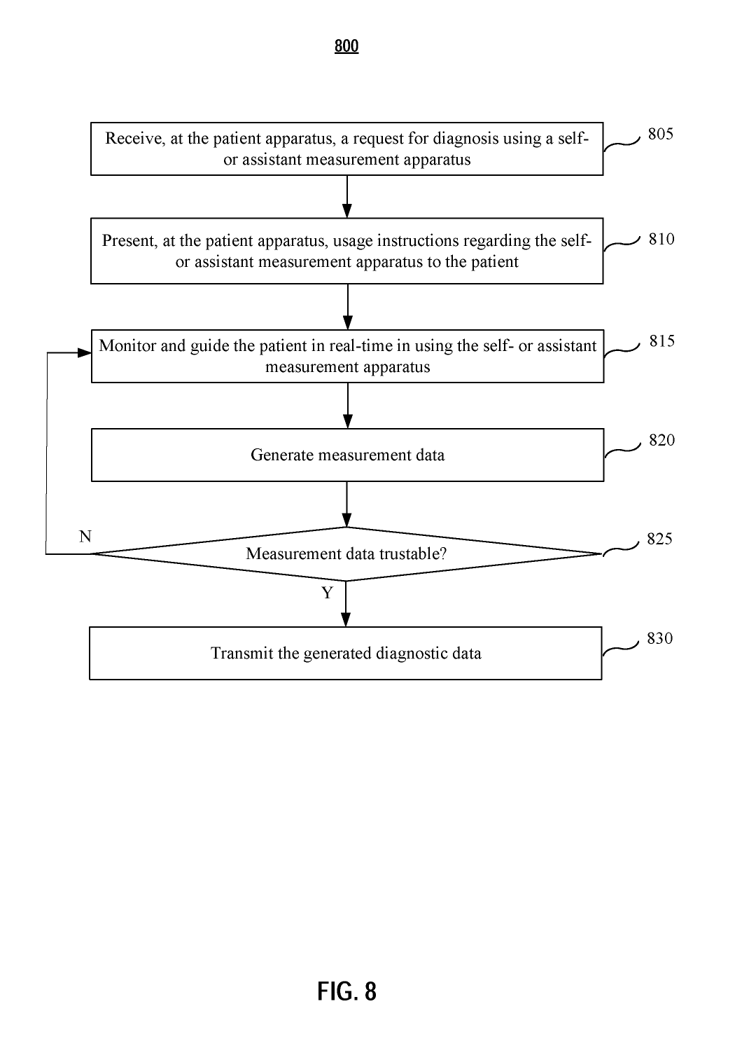

In embodiments, the generated one or more questions by the computing apparatus may be a requirement for the patient to do some self-measurement diagnostics, which may be done without involvement of a healthcare provider. FIG. 8 shows an exemplary flow diagram illustrating a process of self-measurement or assistance measurement by the patient according to various embodiments of the present application.

At step 805, a request for diagnosis using a self- or assistant measurement apparatus is received at the patient apparatus. The request may be sent from a computing apparatus or a doctor apparatus. The request may be presented to the patient adaptively based on medical history information of the patient. For example, the request may be presented in a text, audio, video, augmented or virtual reality or a combination thereof.

In embodiments, the measurement apparatus is a self-measurement apparatus, such as a heart rate sensors, otoscopes, a digital stethoscopes, an in-ear thermometers, a blood oxygen sensor, a high-definition camera, a spirometers, a blood pressure meter, a respiration sensor, a skin resistance sensor, a glucometer, an ultrasound devices, an electrocardiographic sensor, a body fluid sample collector, a weight scale, or any devices known in the art that may be self-operated or assistant operated in performing medical diagnosis. In embodiments, patient characteristics and vital signs data may be received from and/or compared against wearable or implantable monitoring devices that gather sample data, e.g., a fitness device that monitors physical activity. In embodiments, the self- or assistant measurement apparatus may be integrated within the patient apparatus or a stand-alone apparatus coupled to the patient apparatus. The self- or assistant measurement apparatus may contact a patient's body via patches or electrodes for good physical or electrical contact, or may perform contact-less measurements some distance away from the patient's body.

At step 810, when the patient accepts the request, usage instruction of the self- or assistant measurement apparatus is presented to the patient. The instruction may comprise location of the apparatus, operation procedures, etc. The instruction may be presented in a text, audio, video, or an interactive stream format.

At step 815, the patient is guided and monitored in real-time in using the self- or assistant measurement apparatus. The guidance and monitoring may be enabled by using a camera, an accelerometer, a multi-axis gyroscope, a pressure sensor, and/or a magnetometer to provide position, motion, pressure, orientation of the self-measurement apparatus based on patient interaction. In embodiments, a virtual reality (VR) algorithm is implemented for the guidance and monitoring. In embodiments, once the self-measurement apparatus is ready for measurement, a notice may be presented on the patient apparatus. The notice may be an icon, a voice message, a vibrating signal, or a combination thereof.

At step 820, the measurement apparatus begins the measurement and measurement data are generated. The data may be a digital image, a measurement reading, a video, etc.

At step 825, a preliminary trustability check is performed to the generated measurement data. In embodiment, the measurement data is compared against a pre-determined range for trustability check. For example, a measured heartbeat rate should be within a reasonable range, such as between 40 and 120 beats per minute. A measured rate lower than 30 or higher than 200 may indicate a false measurement. The preliminary trustability check may be a location check to ensure the measurement was actually taken within a certain area. The preliminary trustability check may also be an image characteristic check to ensure certain image characteristics to be present in the measurement image.

If the preliminary trustability check passed, the process goes to step 830, in which the generated data are transmitted to the computing apparatus or the doctor apparatus. Otherwise, the process goes back to step 815 with a new round of measurement guidance and monitoring for another measurement trial.

In embodiments, if a second measurement attempt still fails to pass the preliminary trustability check, a warning message may be sent to a healthcare provider or an apparatus operator for checkup.

Although FIG. 4-FIG. 8 show steps in individual process of the automatic healthcare service, one skilled in the art shall recognize that: (1) certain steps may optionally be performed; (2) steps may not be limited to the specific order set forth herein; (3) certain steps may be performed in different orders; (4) certain steps in FIG. 4-FIG. 8 may be done concurrently; and (4) certain steps in FIG. 4-FIG. 8 may be cross-linked.

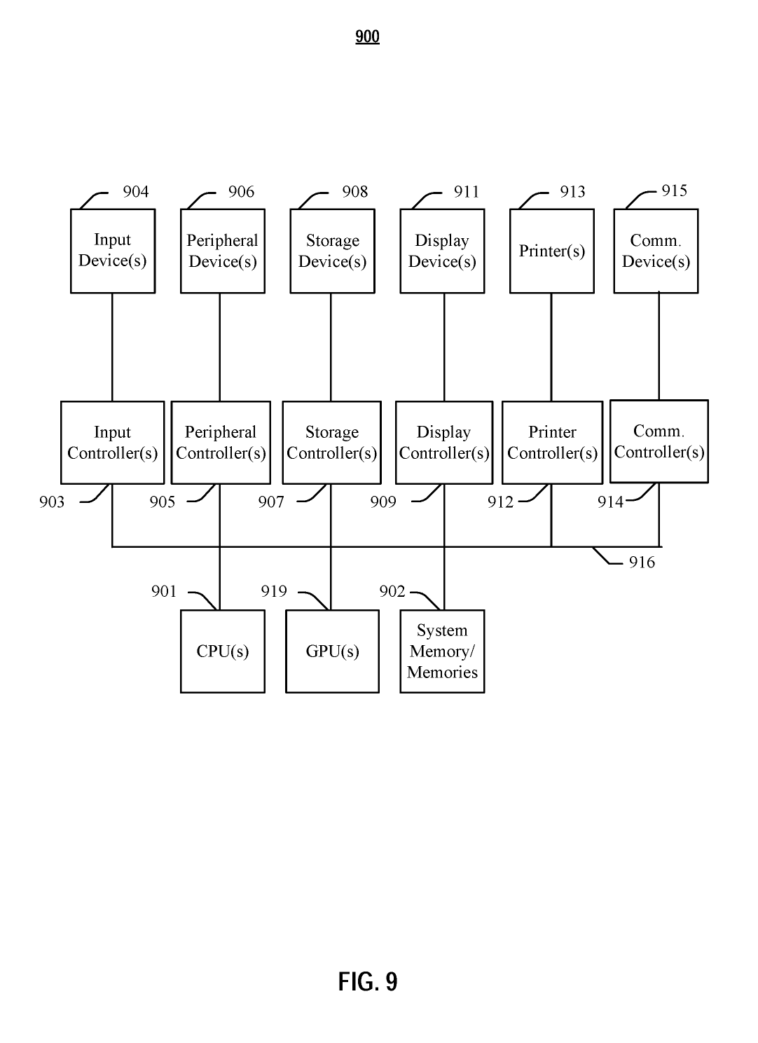

In embodiments, aspects of the present patent document may be directed to, may include, or may be implemented on one or more computing systems. A computing system may include any instrumentality or aggregate of instrumentalities operable to compute, calculate, determine, classify, process, transmit, receive, retrieve, originate, route, switch, store, display, communicate, manifest, detect, record, reproduce, handle, or utilize any form of information, intelligence, or data. An exemplary system that may be used to complement one or more aspects of the present disclosure is described with referent to FIG. 9. Each of the aforementioned patient apparatus, doctor apparatus, computing apparatus, patient management server, usage/billing server, and/or self-measurement apparatus may comprise one or more components in the system 900. It will be understood that the functionalities shown for system 900 may operate to support various embodiments of a computing system—although it shall be understood that a computing system may be differently configured and include different components, including having fewer or more components as depicted in FIG. 9.

As illustrated in FIG. 9, the computing system 900 includes one or more central processing units (CPU) 901 that provides computing resources and controls the computer. CPU 901 may be implemented with a microprocessor or the like, and may also include one or more graphics processing units (GPU) 919 and/or a floating-point coprocessor for mathematical computations. System 900 may also include a system memory 902, which may be in the form of random-access memory (RAM), read-only memory (ROM), or both.

A number of controllers and peripheral devices may also be provided, as shown in FIG. 9. An input controller 903 represents an interface to various input device(s) 904, such as a keyboard, mouse, touchscreen, and/or stylus. The computing system 900 may also include a storage controller 907 for interfacing with one or more storage devices 908 each of which includes a storage medium such as magnetic tape or disk, or an optical medium that might be used to record programs of instructions for operating systems, utilities, and applications, which may include embodiments of programs that implement various aspects of the present invention. Storage device(s) 908 may also be used to store processed data or data to be processed in accordance with the invention. The system 900 may also include a display controller 909 for providing an interface to a display device 911, which may be a cathode ray tube (CRT), a thin film transistor (TFT) display, organic light-emitting diode, electroluminescent panel, plasma panel, or other type of display. The computing system 900 may also include one or more peripheral controllers or interfaces 905 for one or more peripherals 906. Examples of peripherals may include one or more printers, scanners, input devices, output devices, sensors, and the like. A communications controller 914 may interface with one or more communication devices 915, which enables the system 900 to connect to remote devices through any of a variety of networks including the Internet, a cloud resource (e.g., an Ethernet cloud, an Fiber Channel over Ethernet (FCoE)/Data Center Bridging (DCB) cloud, etc.), a local area network (LAN), a wide area network (WAN), a storage area network (SAN) or through any suitable electromagnetic carrier signals including infrared signals.

In the illustrated system, all major system components may connect to a bus 916, which may represent more than one physical bus. However, various system components may or may not be in physical proximity to one another. For example, input data and/or output data may be remotely transmitted from one physical location to another. In addition, programs that implement various aspects of the invention may be accessed from a remote location (e.g., a server) over a network. Such data and/or programs may be conveyed through any of a variety of machine-readable medium including, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices.

Aspects of the present invention may be encoded upon one or more non-transitory computer-readable media with instructions for one or more processors or processing units to cause steps to be performed. It shall be noted that the one or more non-transitory computer-readable media shall include volatile and non-volatile memory. It shall be noted that alternative implementations are possible, including a hardware implementation or a software/hardware implementation. Hardware-implemented functions may be realized using ASIC(s), programmable arrays, digital signal processing circuitry, or the like. Accordingly, the “means” terms in any claims are intended to cover both software and hardware implementations. Similarly, the term “computer-readable medium or media” as used herein includes software and/or hardware having a program of instructions embodied thereon, or a combination thereof. With these implementation alternatives in mind, it is to be understood that the figures and accompanying description provide the functional information one skilled in the art would require to write program code (i.e., software) and/or to fabricate circuits (i.e., hardware) to perform the processing required.

It shall be noted that embodiments of the present invention may further relate to computer products with a non-transitory, tangible computer-readable medium that have computer code thereon for performing various computer-implemented operations. The media and computer code may be those specially designed and constructed for the purposes of the present invention, or they may be of the kind known or available to those having skill in the relevant arts. Examples of tangible computer-readable media include, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices. Examples of computer code include machine code, such as produced by a compiler, and files containing higher level code that are executed by a computer using an interpreter. Embodiments of the present invention may be implemented in whole or in part as machine-executable instructions that may be in program modules that are executed by a processing device. Examples of program modules include libraries, programs, routines, objects, components, and data structures. In distributed computing environments, program modules may be physically located in settings that are local, remote, or both.

One skilled in the art will recognize no computing system or programming language is critical to the practice of the present invention. One skilled in the art will also recognize that a number of the elements described above may be physically and/or functionally separated into sub-modules or combined together.

It will be appreciated to those skilled in the art that the preceding examples and embodiments are exemplary and not limiting to the scope of the present disclosure. It is intended that all permutations, enhancements, equivalents, combinations, and improvements thereto that are apparent to those skilled in the art upon a reading of the specification and a study of the drawings are included within the true spirit and scope of the present disclosure. It shall also be noted that elements of any claims may be arranged differently including having multiple dependencies, configurations, and combinations.

The foregoing description of the present application has been described for purposes of clarity and understanding. It is not intended to limit the present application to the precise form disclosed. Various modifications may be possible within the scope and equivalence of the appended claims.

It will be appreciated to those skilled in the art that the preceding examples and embodiments are exemplary and not limiting to the scope of the present application. It is intended that all permutations, enhancements, equivalents, combinations, and improvements thereto that are apparent to those skilled in the art upon a reading of the specification and a study of the drawings are included within the true spirit and scope of the present application.

It shall also be noted that elements of the claims, below, may be arranged differently including having multiple dependencies, configurations, and combinations. For example, in embodiments, the subject matter of various claims may be combined with other claims.

We claim:

1. A method for automated healthcare service, the method comprising:
receiving input from a patient via a patient interface at a patient apparatus, the input comprising patient identification data and symptom data;
retrieving medical history information from an electronic health record (EHR) database based on the input from the patient;
retrieving reference information based on the symptom input from the patient and the retrieved medical history information;
implementing a trustability analysis for the retrieved reference information to select reference information from the retrieved reference information based at lease on the trustability analysis;
analyzing, using a diagnostic module at a computing apparatus, the input from the patient, the retrieved medical history information and selected reference information, to generate diagnostic data.

2. The method of claim 1, wherein the reference information is retrieved from both medical related resource and non-medical related resource.

3. The method of claim 1, wherein medical related resource comprises external EHR database, medical insurance database, or medical knowledge database.

4. The method of claim 1, wherein the trustability analysis comprising:
assigning a trustability factor to each retrieved information according to at least information source, information published data, or information citation times;
ranking all retrieved reference information according to the assigned trustability factor; and
selecting top N ranking information as the selected reference information, N being an integer number larger than 1.

5. The method of claim 4, wherein the trustability analysis comprising
implementing a conflict analysis to identify a conflict involving conflicted reference information among the retrieved reference information; and
solving the conflict by removing one or more pieces of conflicted reference information from the identified conflicted information.

6. The method of claim 5, wherein the removed one or more conflicted reference information has lower trustability factor than remaining conflicted reference information.

7. The method of claim 1, wherein the retrieved information is mapped according to one or more symptom parameters, and categorized to presented the retrieved information coherently.

8. A system for automated healthcare service, the system comprising:
a patient apparatus comprising a patient interface to receive patient input comprising patient identification data and symptom data;
an electronic health record (EHR) database coupled to the patient apparatus for retrieving medical history information related to the patient based on the patient input;
a medical information communication application (MICA) module coupled to the patient apparatus and the EHR database, the MICA module retrieves reference information based on the patient input and the retrieved medical history information; and
a computing apparatus coupled to receive the patient input, the retrieved medical history information, and the retrieved reference information, the computing apparatus comprising a diagnostic module to analyze the received information for diagnosis;
wherein in response to additional patient input needed based on the analysis, the computing apparatus generates a request for additional patient input, the request is transmitted to the patient apparatus for the patient to respond;
wherein in response to no additional patient input needed based on the analysis, the computing apparatus generates one or more diagnostic results.

9. The system of claim 8, wherein the request for additional patient input is adaptively presented to the patient based on the retrieved medical history information related to the patient.

10. The system of claim 9, wherein the request for additional patient input is adaptively presented in a text format, an audio format, a video format, or a combination thereof.

11. The system of claim 8, wherein the request for additional patient input comprises one or more questions to be answered by the patient, wherein additional patient input responded by the patient is transmitted back to the computing apparatus for a new round of analysis at the computing apparatus.

12. The system of claim 8, wherein the request for additional patient input is a request for using a measurement apparatus to obtain a measurement diagnostic result from the patient.

13. The system of claim 12, wherein the measurement apparatus couples to the patient apparatus, when the patient accepts the request, usage instruction of the measurement apparatus is presented to the patient via the patient apparatus.

14. The system of claim 12, wherein real-time guidance is enabled when the measurement apparatus is used to ensure the measurement diagnostic result is properly obtained.

15. The system of claim 12, wherein the measurement apparatus is a self-measurement apparatus or an apparatus for an untrained assistant to operate.

16. A method for automated diagnosis, the method comprising:
authenticating a patient via a patent interface at a patient apparatus;
receiving symptom input from the patient via the patient interface;
retrieving internal electronic health records (EHRs) from an internal EHR database;
retrieving external EHRs from an external EHR database;
mapping, using a symptom translation module, the retrieved internal EHRs and external EHRs according to one or more symptom parameters to present internal and external EHRs coherently;
retrieving reference information based on at least on the internal EHRs, the external EHRs and the symptom input;
implementing a trustability analysis for the retrieved reference information to select reference information from the retrieved reference information based at lease on the trustability analysis; and
presenting the selected reference information to a computing apparatus with a diagnostic module to start a diagnostic analysis.

17. The method of claim 16, wherein the reference information is retrieved from both medical related resource and non-medical related resource.

18. The method of claim 16, wherein external EHR database comprises EHR database managed by medical insurance, or EHR database managed by third-party healthcare providers.

19. The method of claim 16 further comprising:
generating a request for additional patient input based on the diagnostic analysis;
receiving additional input from the patient in response to the request; and
transmitting the additional patient input back to the computing apparatus for a new round of diagnostic analysis.

20. The method of claim 16, wherein the diagnostic module uses artificial intelligence (AI) integrated algorithm for the diagnostic analysis, the diagnostic module is pre-trained using known medical database before it is deployed.
